(19) United States
(12) Patent Application Publication
(10) Pub. No.: US 2026/0272442 A1
(43) Pub. Date: Sep. 17, 2026

(54) Tape and Suture Construct Assembly

(71) Applicant: Conmed Corporation, Largo, FL (US)

(72) Inventors: Kalifa Clarke, Pinellas Park, FL (US); Peter Miller, Largo, FL (US)

(73) Assignee: Conmed Corporation, Largo, FL (US)

(21) Appl. No.: 19/469,492

(22) PCT Filed: Apr. 4, 2024
(86) PCT No.: PCT/US2024/023059

§ 371 Date: Sep. 26, 2025

Related U.S. Application Data
(60) Provisional application No. 63/457,014, filed on Apr. 4, 2023.

(51) Int. Cl. A61B 17/04; A61B 17/06

(52) U.S. Cl. CPC A61B 17/0401; A61B 17/06166; A61B 2017/0404; A61B 2017/0619

(57) ABSTRACT

A tape and suture construct assembly is provided. The tape and suture construct assembly is configured to be secured at its first end to a looped end of a button implant assembly, such as the Conmed® Infinity™ Adjustable Loop Button, and secure to a tendon, ligament, or tissue graft at its second end by means such as suturing or whipstitching. According to an embodiment, the tape support can be made of a textile reinforcement material and comprise two strands of suture extending therefrom. The first suture strand can be a solid strand. The second suture strand can further comprise a suture loop and a needle portion.

### BACKGROUND

### 1. Field

The present disclosure relates to surgical devices, systems and surgical procedures and, more particularly, to surgical devices, systems and procedures for securing a graft to a button implant assembly.

### 2. Description of the Related Art

Ligament and tendon reconstruction surgeries commonly require the use of a tissue graft to replace the torn ligament or tendon. In these procedures, the graft can be secured to surrounding bone using a button implant assembly by methods including tying, folding over, or directly suturing the graft to the button implant assembly.

Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time, and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### BRIEF SUMMARY

The inventor(s) recognize that there are potential problems and/or disadvantages with the conventional devices and methods for securing a reinforced tissue such as a tendon, ligament, or tissue graft to a button implant assembly in knee reconstruction surgeries. Broken or torn tendons and ligaments can have ends that are weak, making reconstruction difficult as further ripping or tearing of the graft can occur. In cases where a tissue graft is required, the graft must be secured to a button implant assembly but is often too short to wrap around the looped portion of the button implant assembly and too weak to be tied or sutured to the button implant assembly directly. Therefore, a need exists for a simplified and alternative approach for securely attaching a tendon, ligament, or tissue graft to a button implant assembly.

It is therefore a principal object and advantage of embodiments of the present disclosure to provide a tape and suture construct assembly that can be used, at least in part, as a reinforcement material configured to attach a button implant assembly to a graft. The tape and suture construct assembly can be configured to be secured to a loop portion a button implant assembly and secured to an end of a tissue graft by means such as suturing or whipstitching. As described herein, the button implant assembly can include but is not limited to the button assembly as shown and described in US 20200281712 and/or the button assembly as shown and described in U.S. Pat. No. 9,700,403. According to an embodiment, the tape support can be made of a textile reinforcement or suture tape material and comprise two tails of suture extending therefrom. The first suture tail can be a solid strand. The second suture tail can further comprise a suture loop or opening through the suture strand, or can be combined with another opening otherwise connected to the second suture tail, and a needle at the most distal end of the second suture tail.

It is a further object and advantage to provide a method for attaching a tape and suture construct assembly to a tendon, ligament, or tissue graft. A first end of a tape support portion/tape body portion can be secured to (e.g., wrapped around, or a portion of the button implant assembly can otherwise be passed therethrough, or stitched thereto) a portion (e.g., a looped portion) of a button implant assembly that is configured to be implanted into or fixed onto surrounding bone. The tape body portion can then be placed on the tissue graft. A first suture strand extending from the second end (or a side) of the tape body portion can be inserted into a loop of the second suture strand extending from the second end (or a side). A needle end of the second suture strand (positioned opposite the end attached to the tape body portion) can then be used to pierce through a first/top surface of the tape support and the tissue graft. Once the needle pierces and passes through the both the tape body and the tissue graft with the first and second suture strands, the first and second suture strands can be separated underneath the tissue (by removing the first strand from the hole or loop formed by, associated with, or positioned through the second strand) and passed around overtop of the tissue and first/top surface of the tape support. The process can be repeated until the strands have been threaded along at least a portion of or the entire length of the tape support (each suturing (e.g., whipstitch) being separated by a space between the next suturing), forming a traditional whipstitch or suture pattern, as should be understood by a person of ordinary skill in the art in conjunction with a review of this disclosure.

### BRIEF DESCRIPTION OF THE SEVERAL VIEWS OF THE DRAWING(S)

The present disclosure will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments. Reference is now made briefly to the accompanying drawings, in which:

FIG. 1 is a top perspective view of a double tape/folded over portion and suture construct assembly shown attached to a button implant assembly according to an embodiment of the present disclosure.

FIG. 2 is a top perspective view of the tape and suture construct assembly shown attached to a button implant assembly of FIG. 1, and positioned on a tissue graft according to an embodiment of the present disclosure.

FIG. 3 is a top perspective view of a single tape and suture construct assembly with a loop positioned at a first end according to an embodiment of the present disclosure.

FIG. 4 is a top perspective view of a single tape and suture construct assembly with a needle attached to a length of suture and connected to a first end thereof according to an embodiment of the present disclosure.

FIG. 5 is a top perspective view of the tape and suture construct assembly shown attached to a button implant, positioned on a tissue graft, and at the start of forming a suture stitching (e.g., a whipstitch) according to an embodiment of the present disclosure.

FIG. 6 is a top perspective view of the suture tails of the tape and suture construct assembly shown in FIG. 5 being connected to form a whipstitch in a subsequent step according to an embodiment of the present disclosure.

FIG. 7 is a top perspective view of the suture tails of the tape and suture construct assembly shown in FIGS. 5-6 being connected and piercing through the tape portion and the graft to form a whipstitch in a subsequent step according to an embodiment of the present disclosure.

FIG. 8 is a top perspective view of the suture tails of the tape and suture construct assembly shown in FIG. 5 being wrapped around the graft to form another whipstitch according to an embodiment of the present disclosure.

FIG. 9 is a top perspective view of the tape and suture construct assembly shown in FIG. 5 whipstitched to a graft according to an embodiment of the present disclosure.

FIG. 10 is a top perspective view of the tape and suture construct assembly shown attached to a button implant, positioned on a tissue graft, and at the start of forming a suture stitching (e.g., a whipstitch) according to an alternative embodiment of the present disclosure.

FIG. 11 is a top perspective view of the suture tails of the tape and suture construct assembly shown in FIG. 10 being connected to form a whipstitch in a subsequent step according to an alternative embodiment of the present disclosure.

FIG. 12 is a side view of the suture tails of the tape and suture construct assembly shown in FIGS. 10-11 being connected and piercing through the graft to form a whipstitch in a subsequent step according to an alternative embodiment of the present disclosure.

FIG. 13 is a side view of the tape and suture construct assembly shown in FIG. 10 pierced through the graft to form a whipstitch in a subsequent step according to an alternative embodiment of the present disclosure.

FIG. 14 is a top perspective view of the suture tails of the tape and suture construct assembly shown in FIG. 10 being wrapped around the graft to form another whipstitch according to an alternative embodiment of the present disclosure.

FIG. 15 is a top perspective view of the suture tails of the tape and suture construct assembly shown in FIG. 5 being connected to form a whipstitch in a subsequent step according to an alternative embodiment of the present disclosure.

FIG. 16 is a side view of the suture tails of the tape and suture construct assembly shown in FIGS. 10-11 being connected and piercing through the tape portion to form a whipstitch in a subsequent step according to an alternative embodiment of the present disclosure.

FIG. 17 is a side view of the tape and suture construct assembly shown in FIG. 10 pierced through the tape portion to form a whipstitch in a subsequent step according to an alternative embodiment of the present disclosure.

FIG. 18 is a top perspective view of the suture tails of the tape and suture construct assembly shown in FIG. 10 being wrapped around the graft to form another whipstitch according to an alternative embodiment of the present disclosure.

FIG. 19 is a top perspective view of the tape and suture construct assembly shown in FIG. 10 whipstitched to a graft according to an alternative embodiment of the present disclosure.

FIG. 20 is a top perspective view of the tape and suture construct assembly shown attached to a button implant, positioned on a tissue graft, and at the start of forming a suture stitching (e.g., a whipstitch) according to an alternative embodiment of the present disclosure.

FIG. 21 is a top perspective view of the tape and suture construct assembly shown in FIG. 20 with the suture strands crossed under the graft to form a whipstitch in a subsequent step according to an alternative embodiment of the present disclosure.

FIG. 22 is a side view of the suture tails of the tape and suture construct assembly shown in FIGS. 20-21 being connected and piercing through the graft to form a whipstitch in a subsequent step according to an alternative embodiment of the present disclosure.

FIG. 23 is a side view of the tape and suture construct assembly shown in FIG. 20 pierced through the graft to form a whipstitch in a subsequent step according to an alternative embodiment of the present disclosure.

FIG. 24 is a top perspective view of the suture tails of the tape and suture construct assembly shown in FIG. 20 being wrapped around the graft to form another whipstitch according to an alternative embodiment of the present disclosure.

FIG. 25 is a top perspective view of the tape and suture construct assembly shown in FIG. 20 whipstitched to a graft according to an alternative embodiment of the present disclosure.

### DETAILED DESCRIPTION

Aspects of the present disclosure and certain features, advantages, and details thereof, are explained more fully below with reference to the non-limiting examples illustrated in the accompanying drawings. Descriptions of well-known structures are sometimes omitted so as not to unnecessarily obscure the embodiments of the disclosure in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

While embodiments of the present invention have been particularly shown and described with reference to certain exemplary embodiments, it will be understood by one skilled in the art that various changes in detail may be affected therein without departing from the spirit and scope of embodiments of the invention as defined by claims that can be supported by the written description and drawings. Further, where exemplary embodiments are described with reference to a certain number of elements or number/order of steps it will be understood that the exemplary embodiments can be practiced utilizing either less than or more than the certain number of elements or number/order of steps. If elements are shown in a particular Figure discussed below are not specifically identified with respect to that Figure, the elements should be sufficiently identified with respect to at least one other Figure.

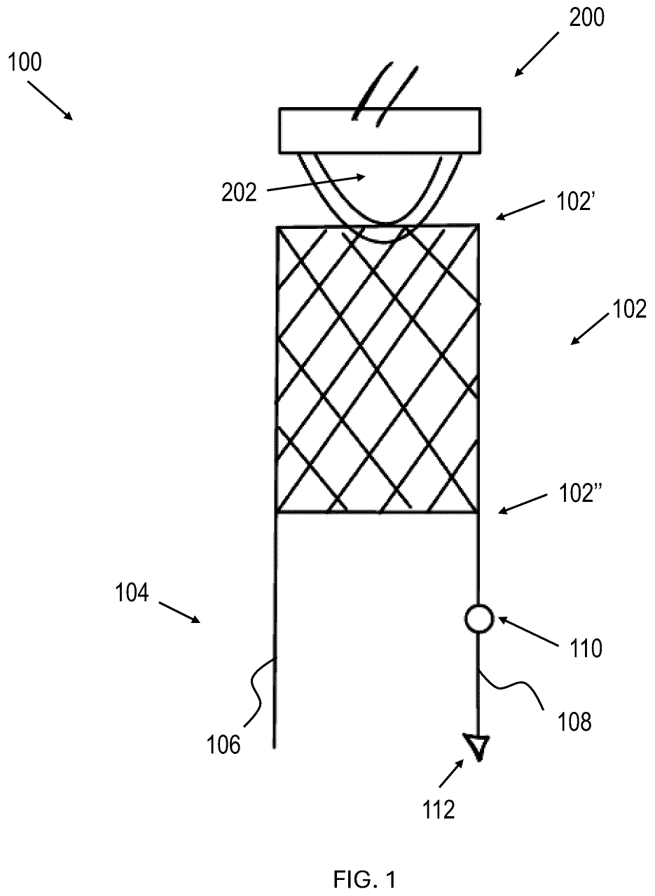

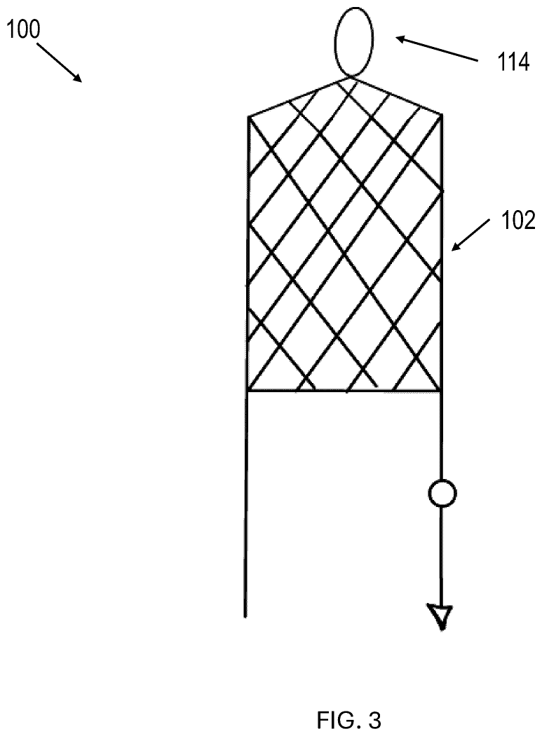

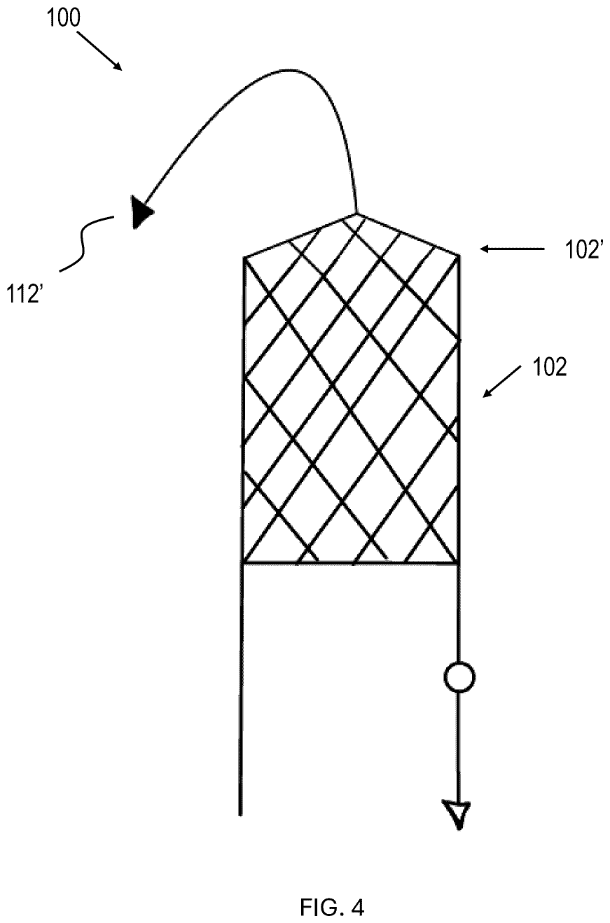

Referring to the figures, wherein like numerals refer to like parts throughout, there is seen in FIG. 1, a double tape and suture construct assembly 100 having a single strand loop and needle configuration. In this configuration, a first end of the tape body portion 102 of the tape and suture construct assembly 100 is doubled/folded over a looped end of a button implant assembly 200 (forming a surgical system including the two assemblies) and is secured to itself by means including but not limited to threading, weaving, tying, or suturing. In some alternative embodiments, the button implant assembly 200 can be joined to the tape and suture construct assembly 100 at the first end 102′ of the tape body portion 102 as discussed further below with respect to FIGS. 3-4. Two suture strands 106 and 108 can extend from the tape and suture construct 100 at the second end 102″ of the tape body portion 102. The first suture strand 106 can be a solid suture strand and the second suture strand 108 can have a needle portion 112 at the opposite end and a looped portion 110 positioned between the needle and the second end 102″ of the tape body portion.

In some embodiments, the needle portion 112 can be a conventional surgical needle. In some embodiments, the looped portion of the second suture strand is positioned on the needle portion itself (not shown). In other embodiments, the looped portion of the first suture strand is formed by creating a loop shape such as an elliptical or diamond with flexible material such as nickel titanium (nitinol) or similar material. In some embodiments, the tape portion 102 can be a flat band of suture or textile material. The suture strands 106 and 108 can be formed of ultra-high molecular weight polyethylene (UHMWPE) or similar material. In some embodiments, the tape is either single or doubled over the piece of tape wrapped around the loop of the button implant assembly, as explained in further detail below.

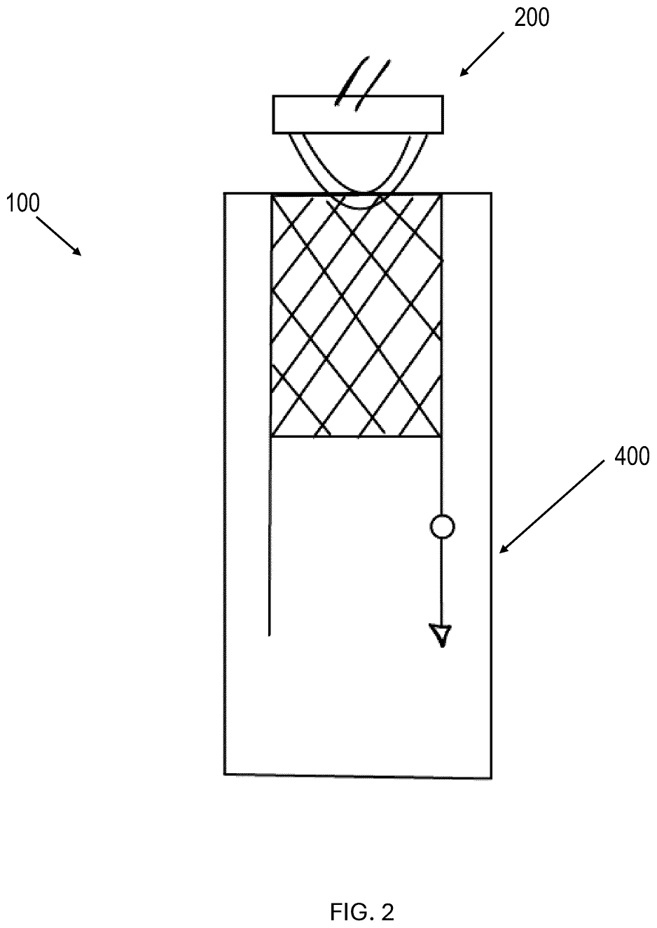

Referring to FIG. 2, the tape and suture construct assembly 100 attached to a button loop assembly 200 of FIG. 1 is shown positioned on a tissue such as a tendon, ligament, or graft 400.

In alternative embodiments, the button loop assembly 200 can be attached to the tape and suture construct assembly 100 (and then to a tendon, ligament, or graft 400) by a variety of other ways. For example, referring now to FIGS. 3 and 4, a single tape (and non-folded over) configuration is shown. In this configuration, the tape body 102 of the tape and suture construct assembly 100 can have a loop 114 attached to the first end 102′ of the tape and suture construct assembly 100 (FIG. 3). The adjustable loop of the button implant assembly (not shown) can be positioned through the loop 114 prior to the button implant assembly being fully formed. Once the button implant assembly is fully formed, the loop 114 and the loop of the button implant assembly can form two closed loops that are intertwined or coupled together. Alternatively, in other embodiments (FIG. 4), the tape 102 can have a second needle portion 112′ connected to a suture strand that is connected to the first end 102′ of the tape body portion 102, wherein the second needle portion 112′ can be threaded through the adjustable loop(s) of a fully formed button implant assembly 200. Once the second needle portion 112′ is threaded through the button implant assembly adjustable loop, the second needle portion 112′ can return, pierce the tape body portion 102, and secure itself to the tape body portion 102 by means including but not limited to threading, weaving, tying, or suturing.

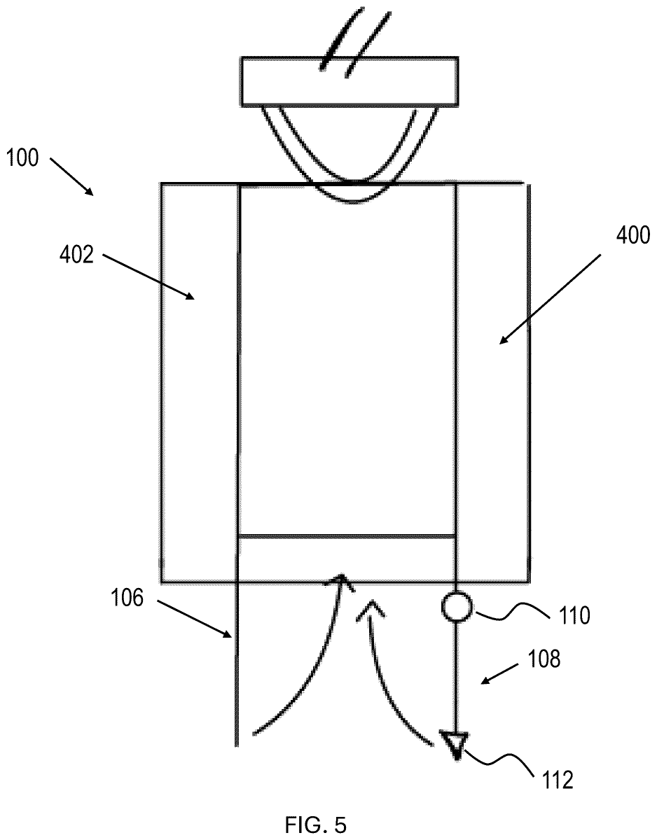

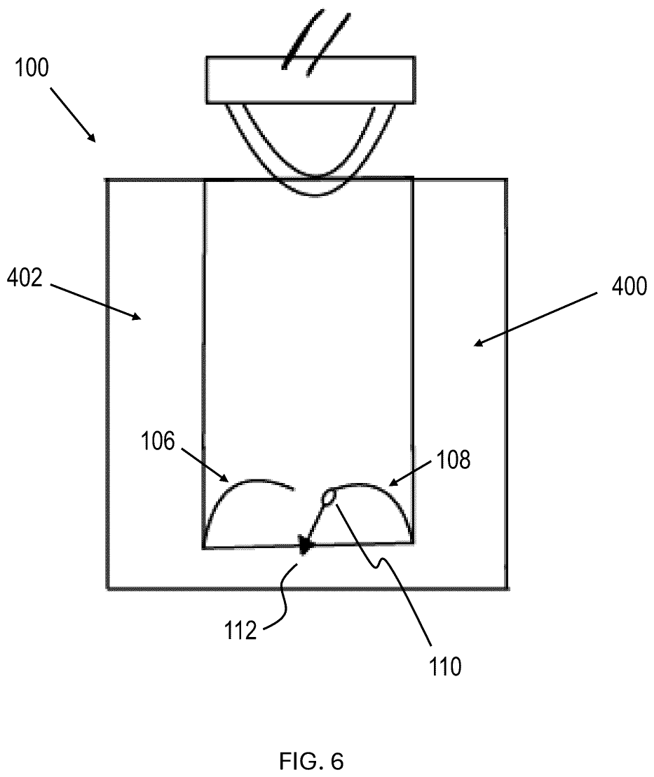

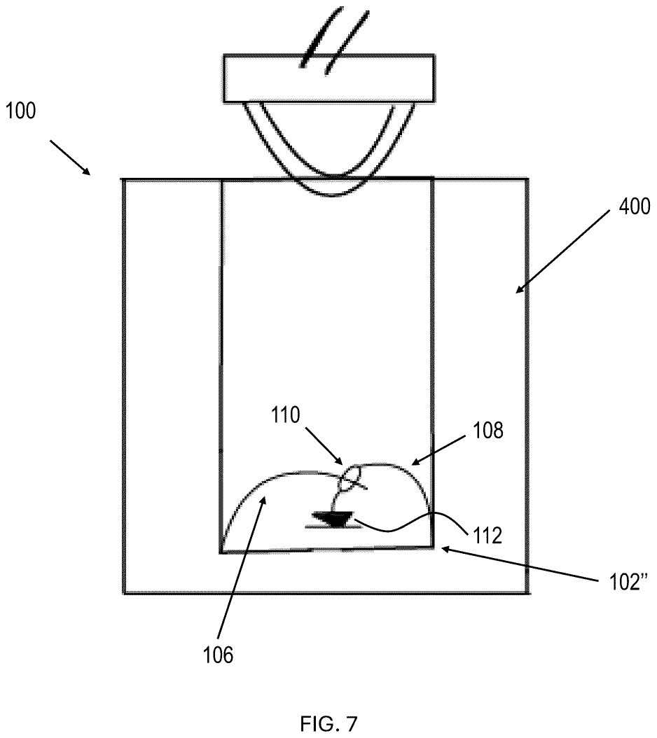

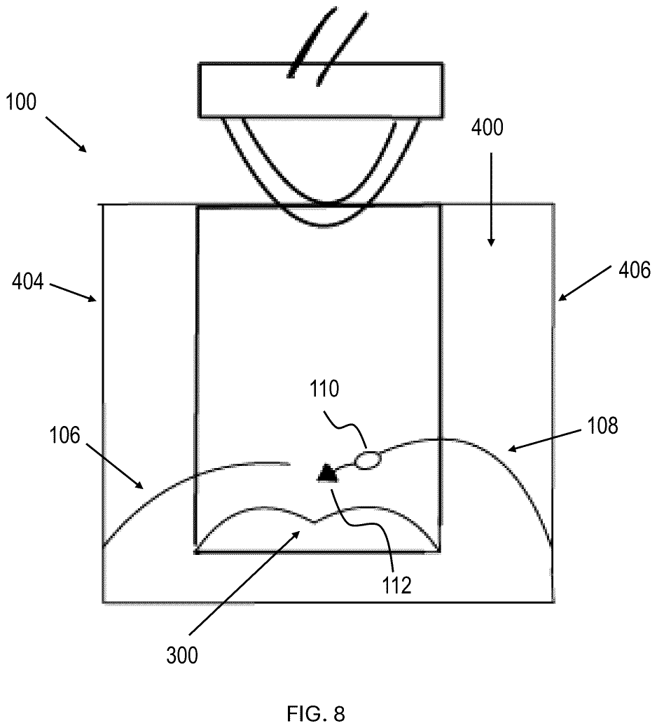

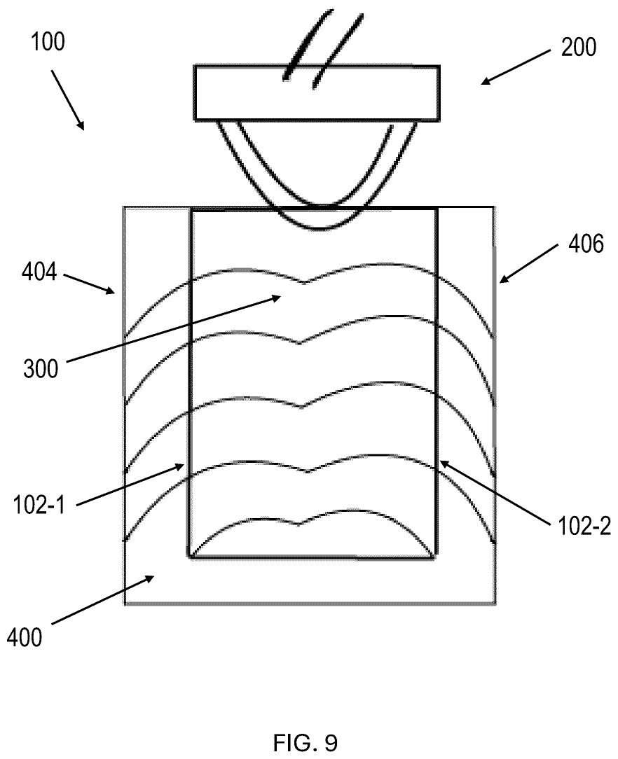

Turning to FIGS. 5-9, a method of attaching the tape and suture assembly 100 with the button assembly 200 (as shown in FIG. 1) to a graft 400 is described. In a first step (FIG. 5), to begin the suturing steps, specifically in this example a whipstitch suture pattern, the suture strands 106 and 108 are brought to be positioned over the tape portion 102 of the tape and suture assembly 100 that is further positioned over the first surface 402 of the graft 400. Next, the first suture strand 106 can be inserted into the loop portion 110 of the second suture strand 108 (FIG. 6). Then, once the first suture strand 106 is inserted into the loop portion 110, the needle portion 112 of the second suture strand 108 can be pierced through the tape portion 102 (at a position adjacent to an end of the tape portion 102, such as the second end 102″, and substantially on or on the central longitudinal axis of the tape body portion 102 (but does not have to be-can be at any point not on the central longitudinal axis)) and the first surface of the graft 402 underneath, causing both the first and second suture strands 106 and 108 to follow therethrough (FIG. 7). Once the needle portion 112 has fully pierced and passed through both the tape portion 102 and the graft 400, the first suture strand 106 can be removed from the loop portion 110 of the second suture strand 108 while positioned underneath the graft 400, and both suture strands 106 and 108 can wrap around the graft 400 on opposite sides, such that one suture strand wraps around the first side of the graft 404 and the other suture strand wraps around the second side of the graft 404 from underneath the graft 400 to above the first surface of the graft 402 (FIG. 8) (e.g., the first suture strand 106 wraps around and passes over the first side of the graft 404 and tape 102-1 and the second suture strand 108 wraps around and passes over the second side of the graft 406 and tape 102-2). This process can be repeated until the suture strands 106 and 108 have been threaded through an entire length of the tape portion 102 (but does not have to be, can be only part of the length, which is less than the entire length), with spaces between each stitch-forming a whipstitch pattern 300 (FIG. 9) as should be understood by a person of ordinary skill in the art in conjunction with a review of this disclosure. This process effectively secures the tape portion 102 of the tape and suture assembly 100 to the graft 400 positioned underneath thereof while the first end of the tape portion 102′ is secured to the button implant assembly 200, as explained above (after which a PCL/ACL/MCL/LCL reconstruction can be finalized, as should be understood by a person of ordinary skill in the art in conjunction with this disclosure). See also FIG. 1.

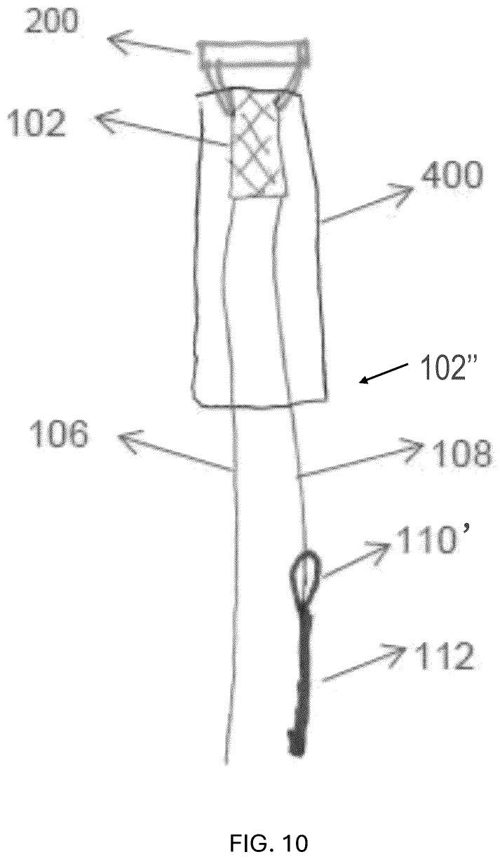

Referring now to FIGS. 10-19, an alternative embodiment of the tape and suture assembly with the button assembly is shown. The assembly configuration and process are substantially the same as described above, with the exception of the structure of the second strand 108. As shown in FIG. 10, for example, the second end of the second strand 108 can alternatively be inserted into and fixed within (e.g., swedging or crimping) a needle portion 112. A separate loop of material or looped strand 110′ can be inserted into and fixed within (e.g., swedging or crimping) the needle portion 112, as shown in FIG. 10. Looped strand 110′ can be formed of a flexible material such as nitinol or a similar material. This embodiment can follow the process of attaching the tape and suture assembly to the graft as described above wherein the first strand 106 can be passed through the looped strand 110′ and the needle portion 112 can pierce through the tape portion 102 and graft 400 underneath. Once positioned underneath, the first strand 106 can be removed from the looped strand 110′ while underneath the graft 400 and each strand 106 and 108 can be passed around the graft at opposite sides, as described above. This process can be completed once a suture or whipstitch (or more than one, forming a pattern) has been formed.

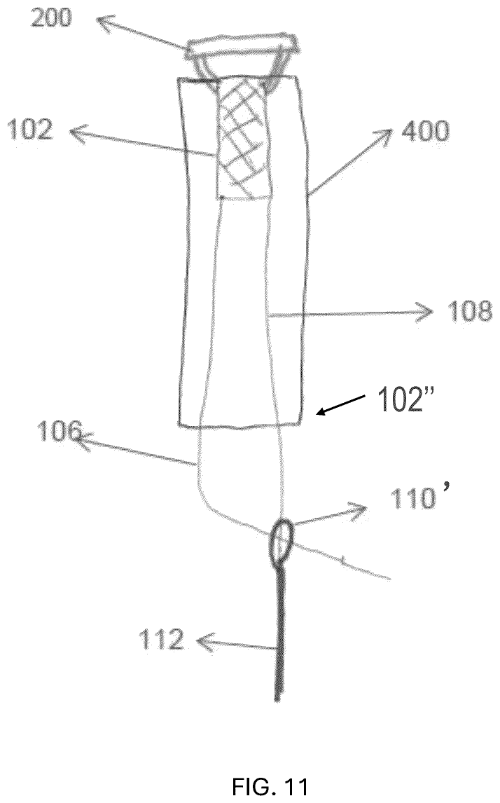

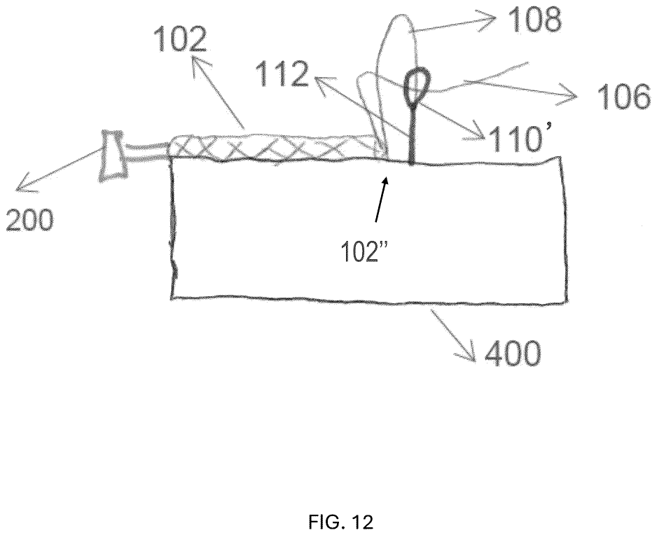

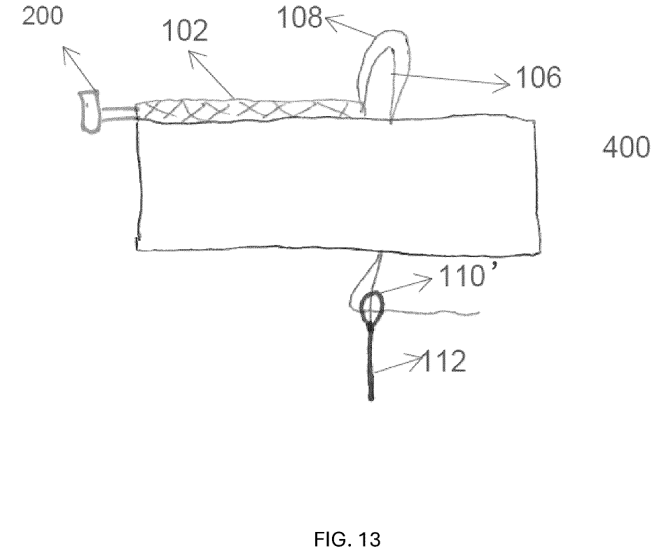

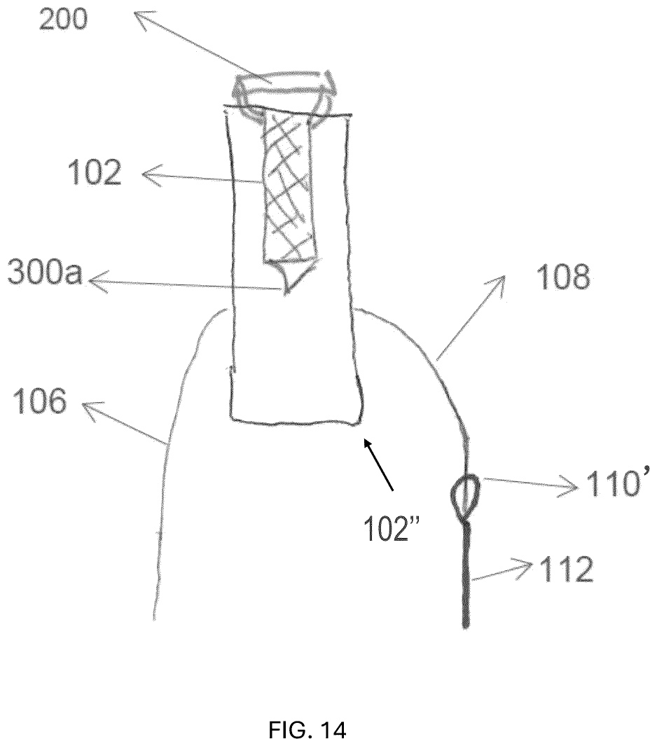

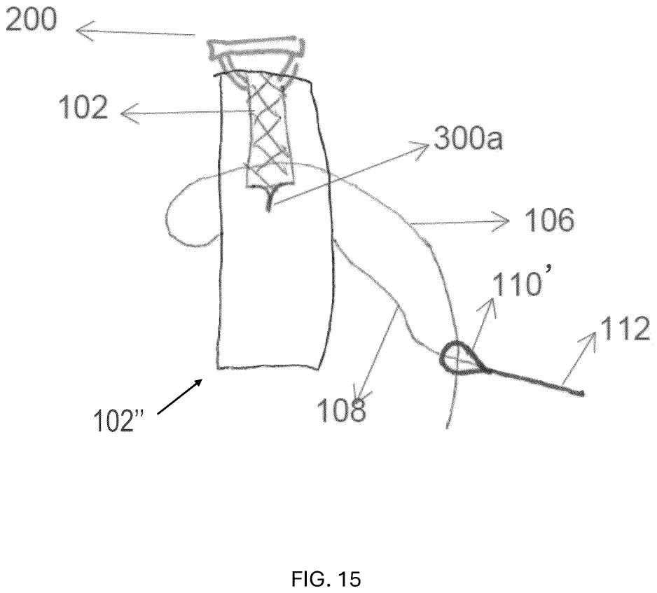

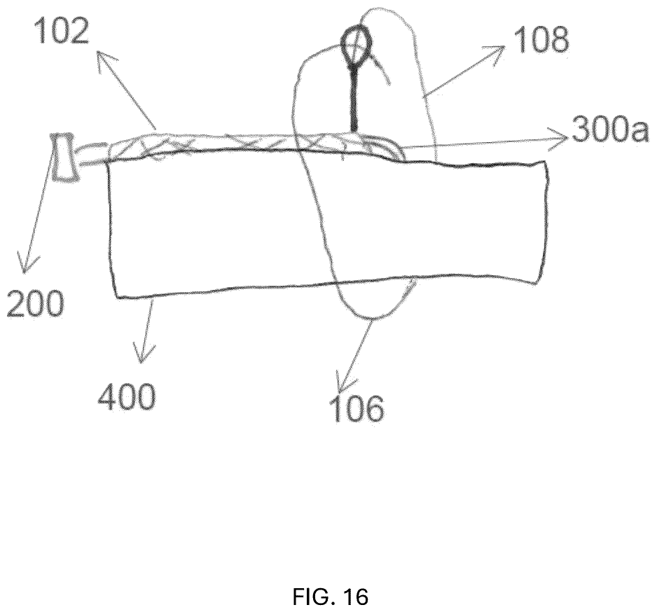

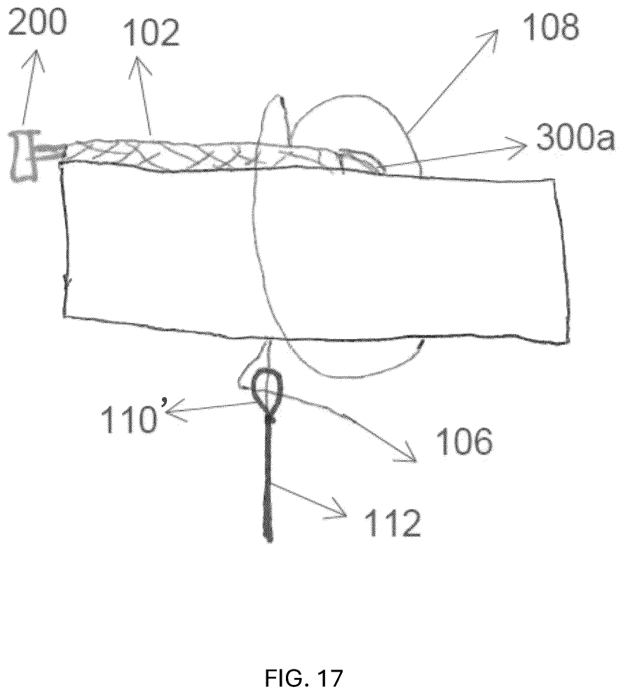

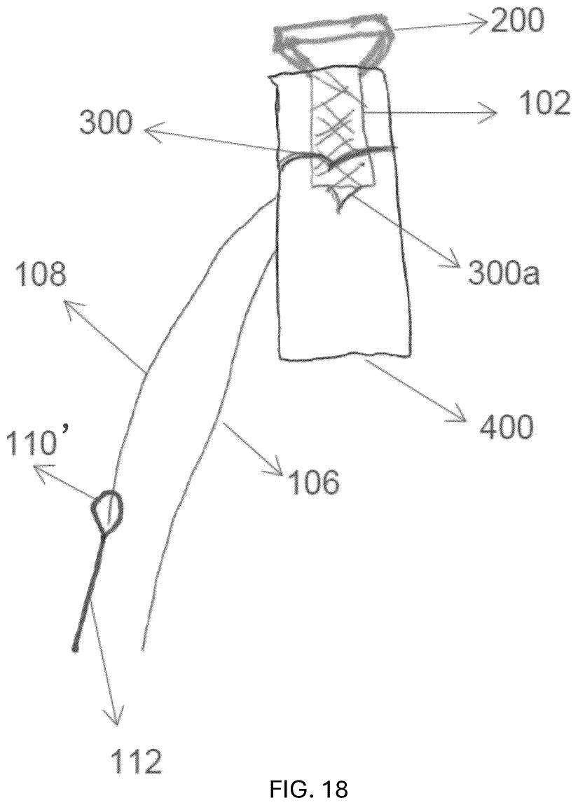

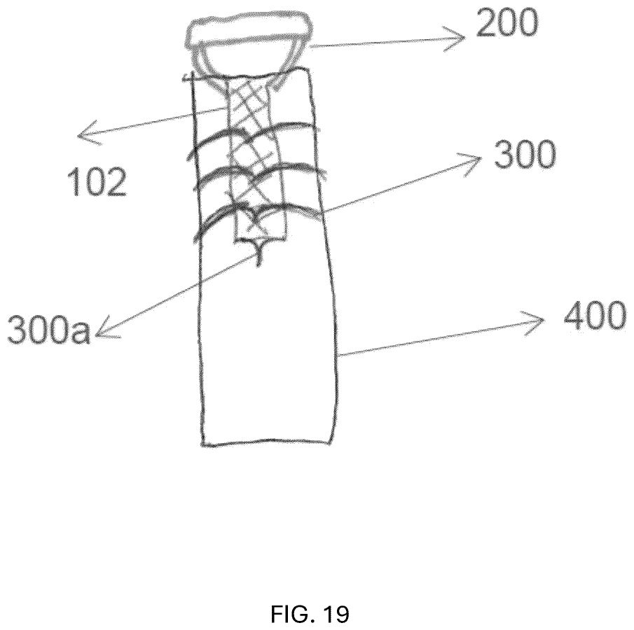

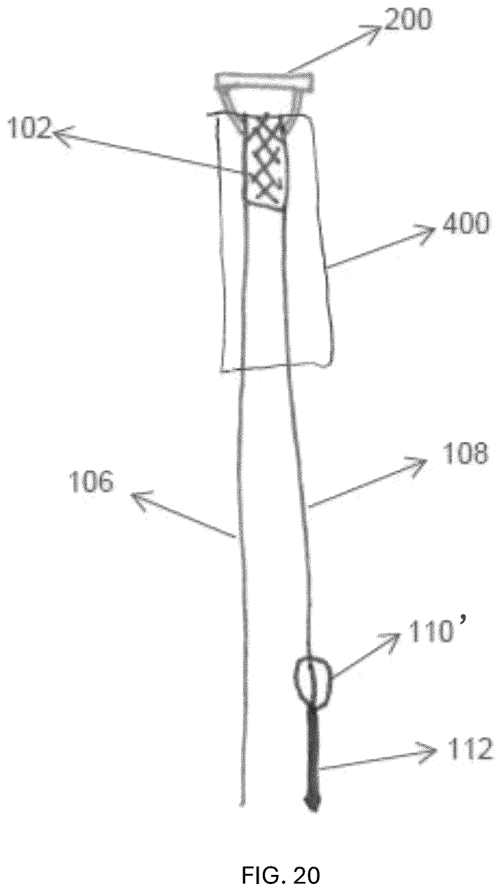

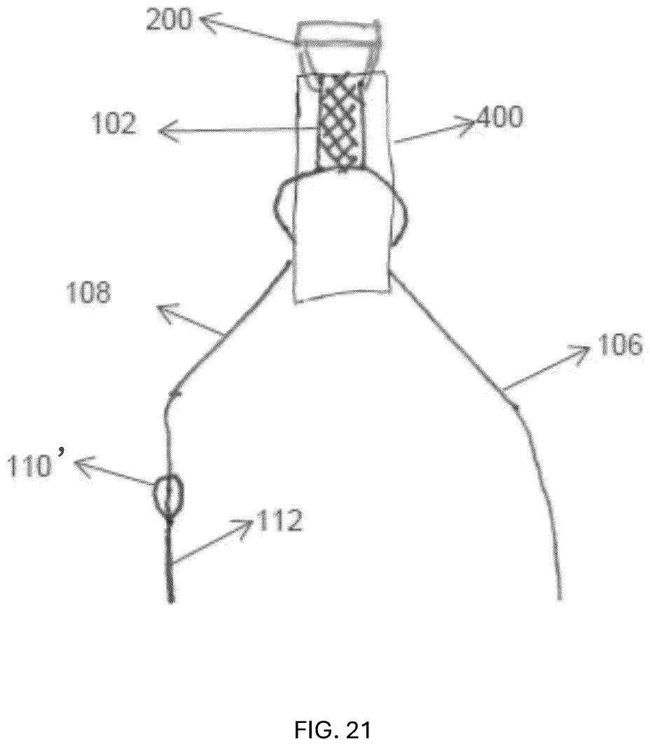

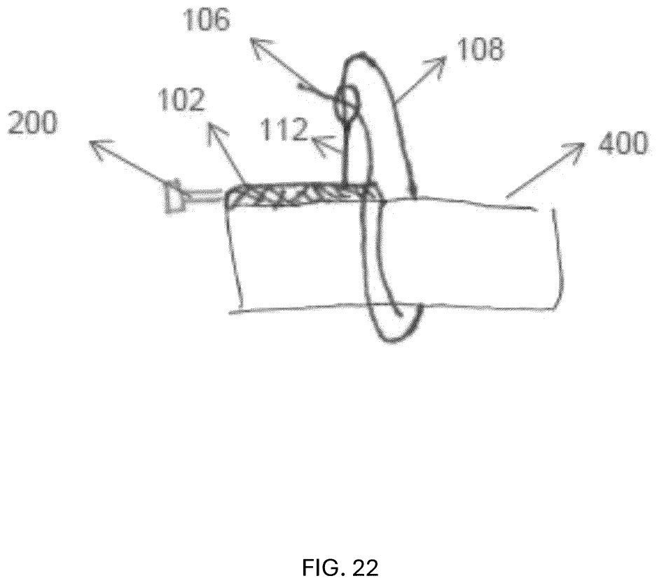

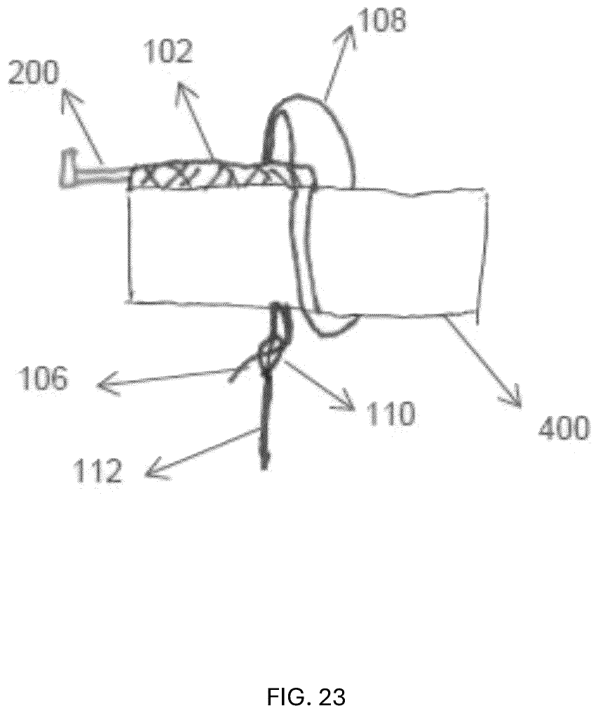

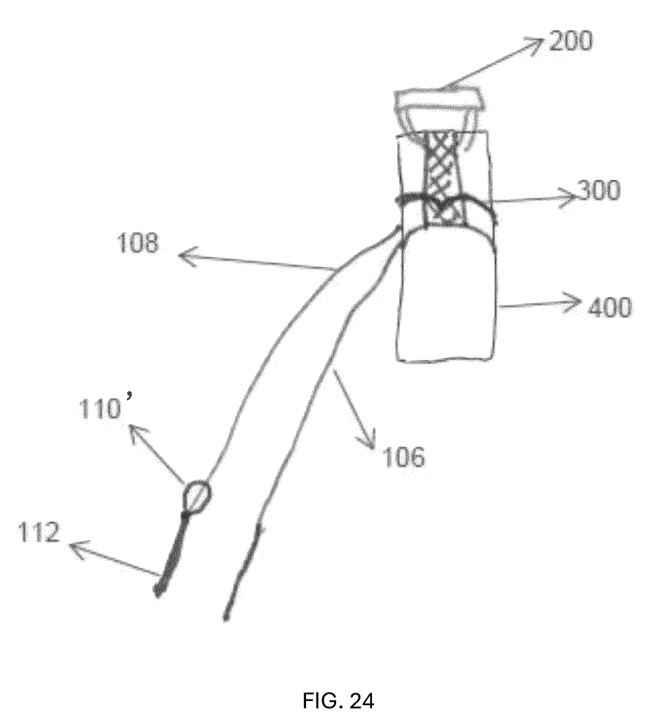

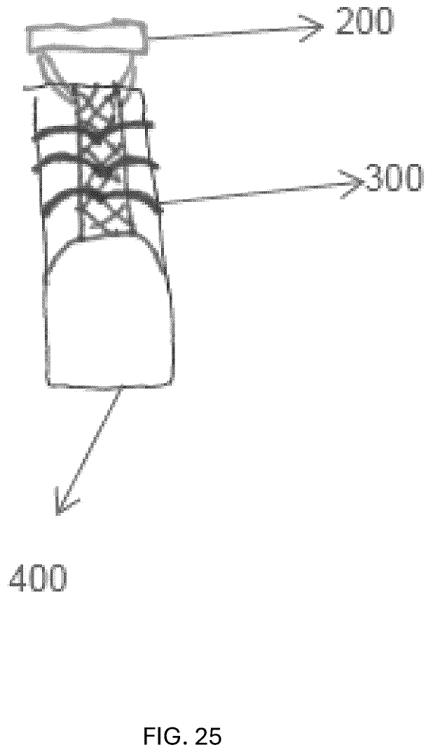

In accordance with the alternative embodiment shown in FIGS. 10-19, the process and the configuration formed pursuant thereof are substantially the same as described above, with the exception of the beginning formation of the whipstitch. In some embodiments, as shown in FIG. 10, to begin the whipstitch, the suture strands 106 and 108 can be brought to be positioned at the base or second end of the tape body portion 102 of the tape and suture assembly 100 that is further positioned over the first surface 402 of the graft 400. Next, the first strand 106 can be passed through the looped strand 110′ (FIG. 11) and the needle portion 112 can pierce the graft 400 at a position 300a adjacent to the second end of the tape portion 102, such as the second end 102″ (FIG. 12). Once the strands 106 and 108 have pierced the graft 400, the strands 106 and 108 can be separated thereunder (FIG. 13) and passed around opposite sides of the graft 400 (FIG. 14). Once positioned above the graft, the first strand 106 can then be passed through the loop formed by the looped strand 110′ (FIG. 15) and the needle portion 112 can pierce both the tape portion 102 (at a position adjacent to an end of the tape portion 102, such as the second end 102″, and substantially on or on the central longitudinal axis of the tape body portion 102) and the first surface of the graft 402 underneath (FIG. 16), causing both the first and second suture strands 106 and 108 to follow therethrough (FIG. 17). The subsequent steps described above can be followed until the suture strands 106 and 108 have been threaded through an entire length of the tape portion 102 (but does not have to be, can be only part of the length, which is less than the entire length) to form a suture or whipstitch pattern as described above (see FIGS. 18 and 19). In some embodiments, as shown in FIGS. 20-25, the first and second suture strands 106 and 108 could be crossed underneath the graft 400 and subsequently brought around opposite sides of the graft 404 and 406 to begin the whipstitch in the first step (FIG. 21). For example, the first suture strand 106 could be crossed underneath the graft 400 at a diagonal such that it emerges at and is wrapped around the second side of the graft 406 while the second suture strand 108 could be crossed underneath the graft 400 at an opposing diagonal such that it emerges at and is wrapped around the first side of the graft 404, creating an X-shape with the suture strands 106 and 108 underneath the graft 400. In this example, the remaining steps could then be repeated to achieve the same or similar result as explained above (See FIGS. 21-25).

In some embodiments, to secure the tape and suture construct assembly 100 to the button implant assembly 200, the first suture strand 106 can be positioned through the second suture strand 108 and the graft 400 can be pierced by the needle portion 112 as explained above. A first set of whipstitches (e.g., two) can then be formed using the steps provided above to secure the tape and suture construct assembly 100 to the graft 400, starting at the second end of the tape portion 102″ and ending at the center of the tape portion 102. Once the first set whipstitches have been formed, the needle portion 112 can be passed through the tape portion 102 and graft 400 underneath with the suture strands 106 and 108 following therethrough. Once both strands 106 and 108 are positioned underneath the graft 400, both strands 106 and 108 can be passed up and through a looped end 202 of the button implant assembly 200 (see FIG. 1) such that the strands 106 and 108 are now positioned over the tape portion 102. A second set of whipstitches (e.g., two) can now be formed following the steps mentioned above, starting at the first end 102′ of the tape portion 102 and ending at a position adjacent to the last of the first set of whipstitches on the tape portion 102, for example. Once the whipstitch pattern is complete, one or more knots can be tied using the strands 106 and 108 to finalize the whipstitch, and the needle portion 112 of the second strand 108 can be pierced through the graft 400, effectively securing the one or more knots within the graft 400 and the tape and suture construct assembly 100 to the button implant assembly 200. In some examples, the needle portion 112 of the second strand 108 (or both strands) can be clipped or cut at a location adjacent to the graft 400 after the knots have been secured within the graft 400 to remove the needle portion from the tape and suture construct assembly 100. In some examples, the second set of whipstitches can form a crisscross or X-pattern over the first set of whipstitches, effectively interlocking the two sets (as should be understood by a person of ordinary skill in the art in conjunction with review of this disclosure).

While the whipstitch suture pattern is discussed above, any suture pattern or technique can be employed (as should be understood by a person of ordinary skill in the art in conjunction with review of this disclosure).

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The terminology used herein is for the purpose of describing particular embodiments only and is not intended to be limiting of embodiments the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as, “has” and “having”), “include” (and any form of include, such as “includes” and “including”), and “contain” (any form of contain, such as “contains” and “containing”) are open-ended linking verbs. As a result, a method or device that “comprises”, “has”, “includes” or “contains” one or more steps or elements. Likewise, a step of method or an element of a device that “comprises”, “has”, “includes” or “contains” one or more features possesses those one or more features but is not limited to possessing only those one or more features. Furthermore, a device or structure that is configured in a certain way is configured in at least that way but may also be configured in ways that are not listed.

The corresponding structures, materials, acts and equivalents of all means or step plus function elements in the claims below, if any, are intended to include any structure, material, or act for performing the function in combination with other claimed elements as specifically claimed. The descriptions of embodiments of the invention have been presented for purposes of illustration and description but is not intended to be exhaustive or limited to embodiments the invention in the form disclosed. Many modifications and variations will be apparent to those of ordinary skill in the art without departing from the scope and spirit of embodiments of the invention. The embodiments were chosen and described in order to best explain the principles of one or more aspects of the invention and the practical application, and to enable others of ordinary skill in the art to understand one or more aspects of the present invention for various embodiments with various modifications as are suited to the particular use contemplated.

What is claimed is:

1. A tape and suture construct assembly, comprising:
a tape body portion having a first end and a second end, a first side and a second side;
a first suture strand having a first end and a second end, wherein the first end of the first suture strand is attached to and extends from the second end of the tape body adjacent to the first side of the tape body;
a second suture strand having a first end and a second end, wherein the first end of the second suture strand is attached to and extends from the second end of the tape body adjacent to the second side of the tape body; and
wherein the second suture strand comprises a needle attached to the second end of the second suture strand and an opening positioned between the needle and the first end of the second suture.

2. The tape and suture construct assembly of claim 1, wherein the opening is positioned through the second suture strand.

3. The tape and suture construct assembly of claim 1, wherein the opening is formed of a loop of material separate from and connected to the second suture strand.

4. The tape and suture construct assembly of claim 3, wherein a portion of each of the loop of material and the second suture is positioned and fixed within the needle.

5. The tape and suture construct assembly of claim 1, further comprising a suture loop extending from the first end of the tape body portion.

6. The tape and suture construct assembly of claim 1, further comprising a third suture strand attached to the first end of the tape body portion.

7. A surgical system, comprising:
a tape and suture construct assembly, comprising:
a tape body portion having a first end and a second end, a first side and a second side;
a first suture strand having a first end and a second end, wherein the first end of the first suture strand is attached to and extends from the second end of the tape body adjacent to the first side of the tape body;
a second suture strand having a first end and a second end, wherein the first end of the second suture strand is attached to and extends from the second end of the tape body adjacent to the second side of the tape body; and
wherein the second suture strand comprises a needle attached to the second end of the second suture strand and an opening positioned between the needle and the first end of the second suture;
button implant assembly, comprising:
a button connected to a first end of a first suture loop, wherein a second end of the first suture loop is connected to the first end of the tape body.

8. The surgical system of claim 7, wherein the opening is positioned through the second suture strand.

9. The surgical system of claim 7, wherein the opening is formed of a loop of material separate from and connected to the second suture strand.

10. The surgical system of claim 9, wherein a portion of each of the loop of material and the second suture is positioned and fixed within the needle.

11. The surgical system of claim 7, wherein the first end of the tape body portion is folded over the second end of the first suture loop.

12. The surgical system of claim 7, further comprising a second suture loop extending from the first end of the tape body portion.

13. The surgical system of claim 12, wherein the second suture loop extends through the first suture loop or the first suture loop extends through the second suture loop.

14. The surgical system of claim 7, further comprising a third suture strand attached to the first end of the tape body portion.

15. The surgical system of claim 14, wherein the third suture strand extends through the first suture loop and is attached to the tape body at a second end via a stitch formed by a needle connected to the second end of the third suture strand.

16. A method of attaching a tape and suture construct assembly to a tissue graft, the method comprising the steps of:
providing a tape and suture construct assembly comprising:
a tape body portion having a first end and a second end, a first side and a second side;
a first suture strand having a first end and a second end, wherein the first end of the first strand is attached to and extends from the second end of the tape body adjacent to the first side of the tape body;
a second suture strand having a first end and a second end, wherein the first end of the second suture strand is attached to and extends from the second end of the tape body adjacent to the second side of the tape body; and
wherein the second strand comprises a needle attached to the second end of the second strand and an opening positioned between the needle and the first end of the second suture;
positioning the tape body on a first surface of the tissue graft;
passing the first suture strand through the opening; and
passing the needle, the first suture strand and the second suture strand through the tape body and the tissue graft.
